(19) United States
(12) Patent Application Publication
(10) Pub. No.: US 2010/0183594 A1
(43) Pub. Date: Jul. 22, 2010

(54) STEROID-SPARING METHODS OF TREATING BRAIN EDEMA

(76) Inventors: Lisa Carr, Calistoga, CA (US); Stephen Evans-Freke, Tuxedo Park, NY (US)

Correspondence Address: JONES DAY, 222 East 41st Street, New York, NY, 10017-6702, US

(21) Appl. No.: 12/560,810

(22) Filed: Sep. 16, 2009

Related U.S. Application Data
(63) Continuation of application No. PCT/US2008/007473, filed on Jun. 16, 2008.
(60) Provisional application No. 61/000,649, filed on Oct. 26, 2007.
(60) Provisional application No. 60/934,910, filed on Jun. 14, 2007.

(51) Int. Cl. A61K 39/395; A61K 38/22; A61P 35/00; A61P 7/10

(52) U.S. Cl. 424/133.1; 514/12

(57) ABSTRACT

The present invention relates to therapeutic regimens or protocols designed for the treatment, management or prevention of edema. In particular, the invention pertains to methods of treating or managing edema associated with brain tumors involving the administration of a therapeutically effective amount of corticorelin acetate that achieves a steroid-sparing effect.

### 1.0 INTRODUCTION

The present invention relates to therapeutic regimens or protocols designed for the treatment, management or prevention of edema. In particular, the invention pertains to methods of treating, managing or preventing edema associated with brain tumors involving the administration of a therapeutically effective amount of corticorelin acetate that achieves a steroid-sparing effect.

### 2.0 BACKGROUND

### 2.1 Edema

Edema, or swelling, is caused by the increase of interstitial fluid in a particular area of the body. Edema can be caused by any disruption of the balance between osmotic pressure and hydrostatic pressure across blood vessel walls. For example, anything that increases oncotic pressure outside blood vessels, such as inflammation, or increases the hydrostatic pressure inside the blood vessel such as heart failure can cause edema. Edema can also be caused by abnormal removal of interstitial fluid caused by obstruction of the lymphatic system due to pressure from a cancer or enlarged lymph nodes, or destruction of lymph vessels by radiotherapy, or infiltration of the lymphatics by infection.

Specifically, brain edema is caused by an increase in brain volume resulting from increased sodium and water content. Brain edema is a common occurrence in patients with malignant and benign brain tumors and is a major cause of morbidity and mortality in such patients. In general there are two categories of brain edema: intracellular edema and extracellular edema.

Intracellular edema is caused by an increase in intracellular water content. This type of edema is often the result of cytotoxic injury such as cerebral ischemia or trauma and is sometimes referred to as “cytotoxic edema.” The primary mechanism of cytotoxic edema is loss of ATP to drive the sodium-potassium ATPase pump causing sodium to accumulate in the intracellular space, creating an osmotic gradient for water to enter the cells.

Extracellular edema is caused by an increase in water in the cerebrospinal fluid or interstitial fluid. There are various types of extracellular edema including vasogenic edema, hydrocephalic edema, osmotic edema, stasis induced by tumor in venous drainage, hydrodynamic processes in which fluid originates from a tumor, and excretory-secretory mechanisms in meningiomas in which tumor-produced substances appear in the peritumoral tissue. Vasogenic edema is the most common type of edema resulting from brain tumors and includes peritumoral and tumoral edema. Vasogenic edema results from an increase in brain capillary permeability causing a pressure gradient from vascular to extracellular compartments to form, allowing fluid to leak into the brain parenchyma.

Hydrocephalic edema is the result of obstruction of cerebrospinal fluid flow. In this type of edema, a hydrostatic gradient between the ventricles and the brain parenchyma is formed. Osmotic edema is the result of an altered osmotic gradient between the plasma and the interstitial fluid. Stasis induced by tumor in venous drainage areas (e.g. compression of an adjacent cortical vein by the tumor), with stasis at the site of the compression results in peritumoral edema.

### 2.2 Treatment of Edema with Steroids

For the past several decades, doctors have treated brain edema with the steroid dexamethasone. Dexamethasone reduces the expression of the edema producing vascular endothelial growth factor (VEGF) which results in a reduction of capillary permeability. Usually, patients suffering from brain edema are exposed to prolonged, high doses of dexamethasone and due to such exposure patients are vulnerable to developing side-effects. Side-effects associated with the administration of high doses of dexamethasone include severe myopathies, muscle wasting, osteoporosis, avascular necrosis, increased appetite, weight gain, gastrointestinal perforation or bleeding, thin or fragile skin, purpura, ecchymoses, inhibition of wound healing, acne, rash, hyperglycemia, redistribution of body fat, peripheral edema and behavioral effects ranging from personality changes and insomnia to psychoses. Furthermore, dexamethasone can interfere with apoptotic death of malignant glioma cells and induce partial resistance to chemotherapeutic drugs and therapeutic irradiation. Often times the side-effects that a patient experiences from the administration of dexamethasone are more debilitating than the neurological effects caused by the brain tumor and the edema, thus researchers have been searching for more effective ways to treat edema that do not cause such debilitating side-effects.

### 2.3 Corticorelin Acetate

Human corticotropin-releasing factor (corticorelin acetate) is an endogenous 41 amino peptide that has been shown to have a peripheral, non-endocrine function mediated biological activity as a potent inhibitor of edema and inflammation (Wei, E. T. et al., Ciba Foundation Symposium 172:258-276 (1993)). This has been confirmed in a series of experiments in which systemic administration of corticorelin acetate has been shown to inhibit vascular leakage of plasma constituents and associated tissue swelling in response to injury or inflammatory mediators (Wei, E. T. et al., European J. of Pharm. 140:63-67 (1987), Serda, S. M. et al., Pharm. Res. 26:85-91 (1992) and Wei, E. T. et al., Regulatory Peptides 33:93-104 (1991)).

With regard to the anti-inflammatory activity of corticorelin acetate, corticorelin acetate prevents vascular leakage induced by a variety of inflammatory mediators that appear to act selectively on post-capillary venules in skin. Corticorelin acetate also inhibits injury- and inflammatory mediator-induced leakage from capillaries in muscle, cerebral micro-vessels, and lung alveolar capillaries. These observations suggest that corticorelin acetate acts throughout the micro-circulation to preserve or restore endothelial cell integrity, thereby inhibiting fluid egress and white blood cell trafficking from the intravascular space and accumulation at sites of injury.

Corticorelin acetate has been shown to be a safe and useful pharmaceutical agent for a variety of different applications in humans. Specifically, in vivo administration of corticorelin acetate has been extensively employed to help elucidate the cause of hyper- and hypo-cortisolemic conditions in humans and is an extremely useful diagnostic and investigative tool for various other disorders affecting the hypothalamic-pituitary-adrenal axis, including endogenous depression and Cushing's disease (Chrousos, G., et al., N. Eng. J. Med. 310:622 (1984) and Lytras, N., et al., Clin. Endocrinol. 20:71 (1984)). In fact, in vivo administration of corticorelin acetate is useful to test corticotropic function of the anterior pituitary in all cases in which an impairment of the anterior pituitary function is suspected. This applies to patients with pituitary tumors or craniopharyngiomas, patients with suspected pituitary insufficiency, panhypopituitarism or empty sella syndrome, as well as patients with traumatic or post-operative injury to the pituitary region and patients who have undergone radiotherapy of the pituitary region. Thus, corticorelin acetate may have utility for diagnostic analysis of the hypothalamus-pituitary-adrenal (HPA) axis.

Studies also have shown that intravenous infusion of corticorelin acetate can reduce or inhibit the development of peritumoral brain edema in rats. Corticorelin acetate was shown to reduce tumoral and peritumoral edema and, administered chronically, significantly prolonged survival over administration of dexamethasone, alone, in rats implanted cerebrally with RG2 glioma tumor cells. Tjuvajev J. et al., Corticotropin-Releasing Factor Decreases Vasogenic Brain Edema, Cancer Res. 1996 Mar. 15; 56(6): 1352-60.

Also, studies have found that intravenous infusion of corticorelin acetate enhanced the anti-edematous effect of dexamethasone in rats. In an in vivo study the anti-edematous effects of dexamethasone alone and in combination with corticorelin acetate were evaluated. In rats bearing intracerebral W256 mammary carcinomas, corticorelin acetate was found to enhance substantially the anti-edematous effect of dexamethasone even at the study's lowest dose of corticorelin acetate. Tjuvajev J, The Effect of hCRF on the Anti-edematous Dose Response Profile of Dexamethasone: an In Vivo Experimental Study, New York, N.Y.: Memorial Sloan-Kettering Cancer Center, 1996.

Additionally, corticorelin acetate has been shown to be well-tolerated in humans. Patients with peritumoral brain edema participating in a phase I trial where they were administered intravenous bolus and continuous infusions of corticorelin acetate showed improvement in neurological symptoms or physical findings. However, intravenous administration of corticorelin acetate did result in some side-effects including headache, hypotension, facial flushing, diarrhea, shortness of breath, nausea and lethargy. Villalona M. A. et al., A Phase I Trial of Human Corticotrophin-releasing Factor (hCRF) in Patients with Peritumoral Brain Edema, Annals of Oncology 9: 71-79, 1998.

Additional experimental and clinical studies indicate that corticorelin acetate may also have a steroid-sparing effect resulting in a reduction in the amount of steroid needed to treat the edema and a reduction in side-effects associated with steroid use. In an ongoing extension study, 20 subjects took corticorelin acetate for at least 4 weeks. Of those subjects, 11 reduced their dexamethasone dosage during the study and 2 took no concomitant dexamethasone. Improvement or resolution of steroid related conditions was observed in 8 of 18 (45%) of subjects with such conditions at baseline. Safety and Steroid-Sparing Potential of XERECEPT™ (corticorelin acetate injection) for Treatment of Peritumoral Brain Edema an Interim Report of an Open-Label Study as Part of a Phase III Program, 11th Annual Meeting of the Society For Neuro-Oncology (SNO) Orlando, Fla. Nov. 15-19, 2006.

All references discussed throughout this specification are herein incorporated by reference in their entirety and no reference cited or discussed is to be construed as prior art.

### 3.0 SUMMARY

The present invention relates to methods of preventing, treating or managing edema. Such methods include therapeutic regimens or protocols designed for the treatment, management or prevention of edema comprising administering corticorelin acetate. The present invention also includes methods of providing steroid-sparing treatments. Certain aspects of the invention include methods of providing a steroid-sparing benefit to a human that is presently receiving a steroid, e.g. corticosteroid or dexamethasone, comprising administering corticorelin acetate. Steroid-sparing benefits include administration of a lower dose of a steroid and the reduction of the side-effects associated with taking the steroid, without comprising the effectiveness of the steroid, including clinical or neurological improvement or stability of the condition for which the steroid is being administered.

The inventors have found that a subcutaneous bolus injection of coticorelin acetate is effective in treating peritumoral edema. This was an unexpected discovery given the short half-life of corticorelin acetate and that the compound was typically administered via continuous infusion. The inventors have found that by administering a subcutaneous injection of corticorelin acetate, alone, to a patient with peritumoral brain edema the edema can be managed or treated and the patient does not experience many of the side-effects associated with steroid therapy. The inventors have also found that by administering a subcutaneous injection of corticorelin acetate to a patient with brain edema who is receiving dexamethasone, the amount of dexamethasone that the patient would have received in the absence of the corticorelin acetate can be reduced. This reduction in dexamethasone results in a reduction in the dexamethasone associated side-effects experienced by the patient.

Accordingly, various aspects of the invention involve the subcutaneous bolus injection of corticorelin acetate for the treatment or management of brain edema. In certain embodiments of the invention, the invention encompasses methods of treating brain edema in a human by administering to a human in need thereof a subcutaneous dose of a therapeutically effective amount of corticorelin acetate.

Other aspects of the invention involve the subcutaneous administration of corticorelin acetate in combination with a steroid, wherein the corticorelin acetate is administered in an amount sufficient to reduce the amount of steroid administered, reduce the frequency of administration of the steroid or reduce the length of time that a patient is exposed to the steroid without compromising the effectiveness of the steroid. In certain embodiments the methods described herein include methods of treating or managing brain edema comprising administering a combination of corticorelin acetate and a steroid, such as dexamethasone, to a human in need thereof, wherein the amount of the steroid in combination with corticorelin acetate is sufficient to treat or manage the edema.

In other embodiments, the methods described herein include methods of treating or managing brain edema comprising administering corticorelin acetate and a steroid to a human in need thereof, wherein the amount of the corticorelin acetate is sufficient to provide the desired effect of the steroid at a lower dose than the amount of the steroid administered in the absence of the corticorelin acetate.

Another aspect of the invention encompasses methods for treating brain edema comprising a treatment regimen comprising administering a steroid in combination with corticorelin acetate, wherein the patients' total exposure to the steroid is reduced by the administration of the corticorelin acetate. The desired effect of the steroid is the prevention, treatment or management of the brain edema which can be evaluated using clinical and neurological tests, examples of which are discussed in further detail below.

The present invention also includes methods of providing a steroid-sparing benefit to a patient that is presently receiving a steroid comprising administering corticorelin acetate to the patient in an amount effective so that the patient can receive a lower dose of steroid and still achieve the therapeutic effect, with reduced side effects.

In certain embodiments the methods described herein include methods for steroid-sparing treatment of brain edema in a human in need thereof comprising administering to the human a combination of a sub-clinically effective amount of steroid and corticorelin acetate, wherein the combination is effective in treating edema.

The present invention also includes methods of reducing the side-effects from a steroid in a human with a treatment regimen comprising administering corticorelin acetate and a steroid, wherein the amount of the corticorelin acetate is sufficient to allow the therapeutic effect of the steroid to be attained with a lower dose of steroid, e.g., half or less of the dose of steroid administered in the absence of the corticorelin acetate.

The present invention also includes methods of dose-sparing of an anti-inflammatory or an anti-cancer agent in a human in need thereof comprising administering to the human an amount of corticorelin acetate, wherein the amount of the corticorelin acetate is sufficient to allow the effect of the anti-inflammatory or an anti-cancer agent to be attained in a lower amount than the amount of the anti-inflammatory or an anti-cancer agent administered in the absence of the corticorelin acetate.

### 4.0 BRIEF DESCRIPTIONS OF THE DRAWINGS

FIG. 1 is a flow diagram depicting the study design of the extension study. The extension study is discussed in further detail in Section 6.0.

FIG. 2 shows dexamethasone dosing over time for all twenty patients enrolled in the extension study.

FIG. 3 is a flow diagram depicting the net cumulative change in dexamethasone dose and steroid-related adverse events for all 20 patients enrolled in the extension study.

### 5.0 DETAILED DESCRIPTION

### 5.1 Definitions

The phrase “steroid-sparing effect” as used herein refers to the effect of a drug, when co-administered with a steroid that allows the dosage of the steroid to be reduced without compromising the beneficial effects of the steroid.

The phrase “steroid taper” as used herein refers to a decrease in the amount of a steroid administered to a patient over a set period of time.

A used herein, a “protocol” includes dosing schedules and dosing regimens. The protocols herein are methods of use.

The terms “treat(s)”, “treated”, “treating” or “treatment” are used herein interchangeably and refer to any treatment of a disorder in an individual diagnosed with the disorder or who has the disorder and has not yet been diagnosed and includes, but is not limited to: (1) caring for an individual diagnosed or inflicted with a disorder; (2) curing or healing an individual diagnosed or inflicted with a disorder; (3) causing regression of a disorder in an individual; (4) relieving, improving, decreasing or stopping the conditions of a disorder in an individual; (5) relieving, decreasing or stopping the symptoms caused by or associated with a disorder in an individual; (6) reducing the frequency, number or severity of episodes caused by or associated with a disorder in an animal; (7) achieving clinical improvements of the cause or conditions of the disorder established by clinical tests; or (8) achieving neurological improvements of the cause or conditions of the disorder established by neurological tests.

The phrase “clinical improvement” used herein refers to a reduction in or resolution of a serious adverse event (“SAE”), an adverse event (“AE”) or clinical complications associated with a disorder or treatment of the disorder including, but not limited to myopathies, muscle wasting, osteoporosis, avascular necrosis, increased appetite, weight gain, bloating, water retention, pancreatis, liver hypertrophy, hyperglycemia, potassium depletion, gastrointestinal perforation or bleeding, thin or fragile skin, purpura, ecchymoses, inhibition of wound healing, acne, rash, hyperglycemia, redistribution of body fat or peripheral edema.

The phrase “neurological improvement” used herein can refer to an increase in neurological test scores or an improvement or a reduction in observed neurological symptoms.

The terms “manage(s)”, “managed”, “managing” or “management” are used herein interchangeably and refer to any treatment of a disorder in an individual diagnosed or inflicted with such disorder and includes, but is not limited to: (1) arresting further development or progression of a disorder in an individual; or (2) slowing the course of a disorder in an individual.

As used herein, the terms “comprising,” “comprises”, “comprised of,” “including,” “includes,” “included,” “involving,” “involves,” “involved,” and “such as” are used in their open, non-limiting sense.

The term “about” as used herein means approximately or near or around. For example, when the term “about” is used in relation to a specified dosage amount or range, the term “about” indicates that the dosage amount or range specified is an approximate dosage amount or range and that it includes not only the amount or range actually specified, but those amounts or ranges that may also be safe and effective amounts that are somewhat outside the cited amount or range.

As used herein, the term “patient” means a human. Additionally, as used herein “patient”, “individual”, “subject” and “human” can be used interchangeably and are not limited to individuals that are under the care of a doctor.

As used herein, the term “steroid-sparing benefit,” refers to a reduction in steroid associated side-effects in a patient receiving a reduced dose of steroid, while still experiencing the therapeutic effect associated with a higher dose of steroid.

As used herein, a “sub-clinically effective amount” is a dosage amount of a therapeutic agent that, if administered alone, would not prevent, manage or treat a disease or condition that it does treat at a therapeutically effective dosage.

As used herein, and unless otherwise specified, a “therapeutically effective amount” means an amount of a compound alone or in combination with another compound sufficient to prevent, treat or manage a disease or condition, or one or more symptoms associated with the disease or condition, or prevent its recurrence. A “therapeutically effective amount” can also encompass an amount of a compound alone or in combination with another compound sufficient to reduce the severity or frequency of a side-effect associated with a disease or condition.

### 5.2 Corticorelin Acetate

Corticorelin acetate is also known in the art as corticotrop(h)in-releasing hormone (CRH), corticoliberin, corticorelin and CRF-41 and is a 41 amino acid peptide that interacts both indirectly with the hypothalamic-pituitary-adrenal (HPA) axis and directly with CRF receptors. Corticorelin acetate is found in high concentrations in the parvocellular (small-celled) neurons of the paraventricular hypothalamic nucleus and influences the HPA axis to ultimately stimulate cortisol production. Corticorelin acetate is also produced directly by peripheral tissues.

The corticorelin acetate neuropeptide was first isolated from extracts of ovine hypothalami (OCRF; Vale, W., et al., Science 213:1394-1397 (1981)) and has subsequently been identified and isolated from the hypothalamus of numerous other mammals including rat (rCRF; Rivier, J., et al., Proc. Natl. Acad. Sci. USA 80:4851-4855 (1983)), porcine (PCRF; Schally, A., et al., Proc. Natl. Acad. Sci. USA 78:5197-5201 (1981) and human (hCRF; Shibahara, S., et al., EMBO J. 2:775-779 (1983)). Comparison of the amino acid sequences of corticorelin acetate peptides from ovine, rat and human has shown that the rat and human peptides are identical, both differing at seven amino acid positions from the ovine peptide, the differences occurring largely in the C-terminal region of the peptides (Hermus, A., et al., J. Clin. Endocrin. and Metabolism 58:187-191 (1984) and Saphier, P., et al., J. Endocrin. 133:487-495 (1993)).

In certain embodiments of the methods described herein the corticorelin acetate is synthetic. For example, an injectable pharmaceutical composition of synthetic corticorelin acetate, identified by the brand name XERECEPT™ is undergoing phase III trials. In certain embodiments of the present invention, the corticorelin acetate used is XERECEPT™. In other embodiments of the methods described herein, derivatives, analogs and conjugates of corticorelin acetate can be used. An example of a conjugate of corticorelin acetate is PEG conjugated corticorelin acetate described in U.S. Application Ser. No. 60/931,786. (filed May 25, 2007, entitled “CRF Conjugates with Extended Half-Lives”, incorporated by reference herein in its entirety).

### 5.3 Methods of Treatment

The present invention relates to methods of preventing, treating or managing edema. Such methods include therapeutic regimens or protocols designed for the treatment, management or prevention of edema comprising administering corticorelin acetate. The present invention also includes methods of providing steroid-sparing treatments. Certain aspects of the invention include methods of providing a steroid-sparing benefit to a human that is presently receiving a steroid comprising administering corticorelin acetate. The present invention also includes methods of providing dose-sparing treatments. Certain aspects of the invention include methods of providing a dose-sparing benefit to a human that is presently receiving an anti-cancer or anti-inflammatory therapy comprising administering corticorelin acetate.

### 5.3.1 Methods of Treating Edema

The present invention is directed to methods of treating or managing edema. The methods described herein include methods of treating or managing edema comprising administering to a patient corticorelin acetate, wherein the corticorelin acetate is administered in an amount sufficient to treat or manage the edema. In some embodiments the treatment results in clinical or neurological improvement of the patient as discussed in Section 5.3.2, infra. Methods described herein include methods for treating brain edema, in a human, by administering to a human in need thereof, one or more subcutaneous bolus doses of a therapeutically effective amount of corticorelin acetate. In certain embodiments, the corticorelin acetate is administered in an amount sufficient to reduce the edema. In other embodiments, corticorelin acetate is administered in an amount sufficient to manage the edema and prevent the edema from escalating i.e. prevent the influx of additional interstitial fluid.

The invention also encompasses methods of treating or managing brain edema that is the result of trauma, injury, toxins or disease to the brain such as traumatic head injuries, craniotomies, blood clots, brain cysts, inflammation or infections. In certain embodiments, the invention encompasses methods of treating or managing edema comprising administering an amount of corticorelin acetate sufficient to reduce inflammation of brain tissue. Methods of measuring inflammation of brain tissue are set forth in Section 5.3.3, infra.

The invention also encompasses methods of treating or managing brain edema due to mechanical, circulatory, osmotic, or metabolic pathologies that cause an expansion of brain volume resulting from an increase in water and sodium content. Such pathologies can result in intracellular or extracellular edema. Types of intracellular and extracellular brain edema that can be treated with the methods described herein include cytotoxic, hydrocephalic, and interstitial and vasogenic types of edema.

Also encompassed by the present invention are methods for preventing edema comprising administering to a patient in need thereof, corticorelin acetate, wherein the corticorelin acetate is administered in an amount sufficient to prevent edema. In such embodiments, patients may be at risk for developing edema. Such patients who are at risk for developing edema can be identified by any method known in the art including the methods discussed in Section 5.3.2, infra. Patients who are at risk for developing edema include patients who have undergone surgery, are recovering from surgery, are currently undergoing surgery or who anticipate undergoing surgery. Patients who are also at risk for developing edema include patients whose homeostatic balance of interstitial fluid has been disrupted as a result of trauma, injury, toxins or disease to the brain such as, but not limited to, traumatic head injuries, craniotomies, blood clots, brain cysts, inflammation or infections. In certain embodiments the invention described herein includes methods of preventing brain edema in a human by administering to a human in need thereof a subcutaneous dose of corticorelin acetate.

In particular, the invention encompasses methods of preventing the development of edema or treating or managing brain edema associated with brain tumors. Such brain tumors can be primary or metastatic. The invention also encompasses methods of preventing, treating or managing brain edema associated with the treatment of brain tumors, including edema associated with side-effects of surgery. As such, the present invention includes methods of treating or managing brain edema in a patient recovering from resection i.e. surgical removal of a brain tumor.

In addition to surgery, the invention also encompasses methods of preventing, treating or managing brain edema associated with side-effects of radiation therapy, chemotherapy, or a combination thereof. Such methods include methods of preventing, treating or managing brain edema in a patient concurrently receiving or recovering from receiving chemotherapy or radiation therapy. For example, one aspect of the invention is a method for treating or managing brain edema in a patient with radiation necrosis.

The invention also encompasses methods of preventing the development of edema or treating or managing brain edema associated with disruption of the blood-brain barrier. Brain edema can be caused by altering the permeability of the blood brain barrier by opening tight junction between endothelial cells, inducing capillary fenestrations, and increasing pinocytic vesicles. Therefore, the invention is also directed to methods of treating or managing edema comprising administering an amount of corticorelin acetate sufficient to rearrange the blood-brain barrier. Rearranging the blood-brain barrier can include retightening the junction between endothelial cells. In still other embodiments, the invention encompasses methods of treating or managing edema comprising administering an amount of corticorelin acetate sufficient to elicit vasoprotective effects.

Other aspects encompassed by the present invention include methods of treating or managing brain edema comprising administering a combination of corticorelin acetate and a steroid. The present invention is based, in part, on the recognition that corticorelin acetate administered subcutaneously can enhance or improve the therapeutic benefit of a steroid for treatment of edema. Thus, the present invention encompasses methods and compositions that comprise administering corticorelin acetate subcutaneously in combination with a steroid. In particular, the invention encompasses treatment regimens and methods of administration that provide i) a better therapeutic profile than that of a steroid administered alone; ii) management of, reduction of, or stabilization of clinical or neurological side effects that may accompany steroid treatment for edema; or iii) reduced exposure to steroids as compared to the exposure to steroid that would have been required the same or better therapeutic results in the absence of corticorelin acetate, as determined by methods known in the art. In a preferred embodiment, the steroid is dexamethasone.

In certain embodiments encompassed by the present invention, methods of treating or managing brain edema comprise administering a combination of corticorelin acetate and a steroid to a human in need thereof, wherein the amount of the steroid in combination with corticorelin acetate is sufficient to treat or manage the edema. In some embodiments of the invention, corticorelin acetate is administered to a patient in combination with a lower dose of a steroid than the steroid dose that would have had to be administered in the absence of the corticorelin acetate, to prevent, manage or treat edema. Administration of lower doses of steroids reduces the risk of the patient developing side-effects associated with steroids. In one embodiment, one or more of the following steroidal side-effects is reduced and/or eliminated: Cushingoid appearance, polyuria, myopathies, oedema peripheral, muscle wasting, osteoporosis, avascular necrosis, increased appetite, weight gain, contusion, visual acuity reduced, abdominal distention, petechiae, compression fracture, bloating, gastrointestinal perforation or bleeding, thin or fragile skin, skin striae, purpura, ecchymoses, inhibition of wound healing, acne, rash, Herpes zoster rash, hyperglycemia, redistribution of body fat, peripheral edema, pedal edema, steroid dependency, and behavioral effects ranging from personality changes and insomnia to psychoses, and inhibition of chemotherapy-induced apoptosis. Therefore, in the methods of treating brain edema described herein, wherein corticorelin acetate is administered with a steroid, a sub-clinical dose of the steroid can be administered to the patient being treated. A sub-clinical steroid dose is a dosage amount of a steroid that, if administered alone, would not prevent, manage or treat edema.

In certain embodiments the methods described herein include methods of treating or managing brain edema comprising administering corticorelin acetate and a steroid to a human in need thereof, wherein the corticorelin acetate is sufficient to allow the effect of the steroid to be attained in a lower amount than the amount of the steroid administered in the absence of the corticorelin acetate. Another aspect of the invention encompasses methods for treating brain edema comprising administration of a treatment regimen comprising a steroid in combination with corticorelin acetate, whereby the total exposure to or frequency of administration of the steroid is reduced by the administration of the corticorelin acetate.

Corticorelin acetate can also be administered as a steroid-sparing agent, therefore, the methods described herein also include methods of treating or preventing edema comprising administering to a patient in need thereof, a treatment regimen comprising corticorelin acetate and a steroid, wherein the corticorelin acetate is administered as a steroid-sparing agent. Corticorelin acetate and a steroid can be administered to a human in need thereof, wherein the corticorelin acetate is administered in an amount that allows the dosage of the steroid to be reduced without compromising the effectiveness of the steroid.

In certain embodiments of the present invention, corticorelin acetate is administered to a patient already on steroid therapy or is administered to a patient concurrently with a steroid. For the treatments described herein that include a steroid, suitable steroids include, but are not limited to, corticosteroids. Corticosteroids include glucocorticoids and mineralocorticoids such as alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, rimexolone, tixocortol, triamcinolone, ulobetasol and combinations thereof.

Side-effects associated with steroids administered at an amount effective to treat edema in absence of the corticorelin acetate can also be reduced. In certain embodiments, the invention described herein includes methods of reducing the severity or the frequency of side-effects associated with administration of a steroid by administering to a patient in need thereof, corticorelin acetate. Additionally, once the corticorelin acetate is administered to such patients, the amount of steroid that the patient receives can be reduced. In certain embodiments, the patient has edema or is already on steroid therapy and/or is at risk of developing edema. Side-effects associated with administration of a steroid are discussed above.

Corticorelin acetate can also be administered to a patient as part of a steroid replacement therapy. In certain embodiments, the present invention includes methods of treating edema comprising administering corticorelin acetate as a steroid replacement therapy. Steroid replacement therapy that includes administration of corticorelin acetate can be administered to refractory patients. For example, the present invention includes administration of corticorelin acetate for the treatment or management of brain edema in patients refractory to steroid therapy. Patients refractory to steroid therapy are patients who have been administered a steroid for the treatment brain edema, and wherein the brain edema being treated i) showed no response to the steroid therapy over a period of time, or ii) did exhibit a response but at some point during the steroid therapy ceased to exhibit a response, or iii) the brain edema proceeded to increase. In certain embodiments the methods described herein include methods for managing brain edema in a patient in need thereof comprising administering to the patient a therapeutically effective amount of a corticorelin acetate wherein the patient does not respond to steroid therapy.

### 5.3.2 Assessing Improvement of Edema

In certain embodiments of the methods described herein, methods of treating edema result in clinical and/or neurological improvements of the edema. Therapeutic benefits of the methods of the invention such as clinical improvement of edema, can be determined by medical imaging such as CT scans and MRI. On CT and T1-weighted MRI, brain edema can be visualized as a hypodense or hyperintense lesion. Brain edema and other structures with a high water content, such as cerebrospinal fluid, are hyperintense on T2-weighted MRI. Fluid-attenuated inversion-recovery MR images provide additive information since brain edema is clearly visualized as a hyperintense lesion against an iso- or hyperintense background. Fluid-attenuated inversion recovery MRI has been compares with T2-weighted MRI in patients with meningiomas and is superior to T2-weighted images with respect to tumor delineation, contrast of tumor to brain parenchyma.

Various methods can be used to display alterations in biochemistry in peritumoral brain edema. Changes in perfusion can be studied with dynamic per-fusion-weighted MRI. Significant reductions in blood flow, oxygen, and glucose in peritumoral edema can be assessed using positron emission tomography. Intraoperative studies can be used to show increases in peritumoral tissue oxygen levels.

Neurological improvements in edema can be determined by an NCI neurological exam, Karnofsky Performance Status (KPS) and mini-mental status examination (MMSE) scores. The NCI neurological exam can be used to check brain, spinal cord, and nerve function by checking a person's mental status, coordination, ability to walk, and how well the muscles, sensory systems, and deep tendon reflexes work. The KPS can be used to measure the ability of patients to perform ordinary tasks. The Karnofsky Performance scores range from 0 to 100. A higher score means the patient is better able to carry out daily activities. Thus, in certain embodiments, treating edema with corticorelin acetate can result in the therapeutic benefit of increasing the patient's KPS score. The MMSE can be used to track a patient's cognitive improvement in five areas of cognitive function: orientation, registration, attention and calculation, recall, and language. The MMSE has a maximum score of 30. Thus, in certain embodiments, treating edema with corticorelin acetate can result in an increase in the patient's MMSE score. In a particular embodiment, the methods of the invention result in the therapeutic benefit of one or more areas of cognitive function as assessed by MMSE.

In certain embodiments of the methods described herein, methods of treating edema in a patient result in an improvement in the patient's performance status, quality of life and/or general wellbeing as measured by any method known in the art. Methods of measuring a patient's performance status are well-known in the art and include quantitative measurements of physical, functional, and symptom control, as well as psychological and social indicators. Scales for measuring quality of life include the Karnofsky Performance Status Scale (discussed above), the Zubrod Scale (ECOG), and the Lansky score. Thus, in certain embodiments, treating edema with corticorelin acetate can result in the therapeutic benefit of increasing the patient's performance status as measured by any quantitative method known in the art.

Also, in certain embodiments described herein, the methods of treating edema result in a reduction in side-effects associated with steroids that a patient experiences. A reduction of in side-effects can include a reduction in the number of side-effects that a patient experiences, a reduction in the severity of a side-effect, a reduction in the duration of a side-effect, or a reduction in the frequency in which a side-effect occurs. Side-effects associated with steroids include those discussed in Section 5.3.1, infra.

### 5.3.3 Steroid-Sparing Treatments

The present invention is also directed to methods of providing a dose-sparing benefit to a human that is presently receiving an agent that is effective in treating edema. In certain embodiments the invention is directed to methods of providing a steroid-sparing benefit to a human that is presently receiving a steroid comprising administering corticorelin acetate. Administration of corticorelin acetate to a patient that is currently receiving a steroid or who is on steroid therapy, allows the patient to reduce the level of steroid that the patient is exposed to without reducing the beneficial effects of the steroid and, at the same time, reduce the severity, frequency, duration, or number of side-effects associated with the steroid that the patient experiences.

For example the present invention encompasses methods of providing a steroid-sparing benefit to a human that is presently receiving a steroid comprising administering corticorelin acetate, wherein the amount of the corticorelin acetate is sufficient to allow the effect of the steroid to be attained in a reduced amount than the amount of steroid administered in the absence of the corticorelin acetate. For example, patients receiving 4 mg/d of dexamethasone or other steroid can be administered an amount of corticorelin acetate such that the amount of dexamethasone that the patient received is reduced to 3 mg/d or less without compromising the effect of the steroid. In one embodiment, a patient who is undergoing steroid therapy who is administered corticorelin acetate in an amount to effect steroid-sparing will eventually receive no steroid. In one embodiment of the invention, the steroid is dexamethasone. It is not uncommon for dexamethasone to be administered in high doses, such as 16 mg/day. Dexamethasone may also be administered at doses of 40 mg/day. Accordingly, an object of the invention is administration of corticorelin acetate to a patient receiving a high dose of dexamethasone (e.g., 20 or 40 mg/day for 4 days out of the month) in an amount such that the dose of dexamethasone administered to the patient can be tapered. Accordingly, one embodiment of the invention encompasses the administration of corticorelin acetate to a patient who is receiving dexamethasone at a baseline dosage of 16-40 mg/day, wherein subsequent dosages of dexamethasone decrease over a given time interval. For example, in one embodiment of the invention, corticorelin acetate is administered to a patient who is receiving 16 mg/day of dexamethasone. Accordingly, the patient is administered 16 mg/day of dexamethasone for a two day period, followed by administration of 14 mg/day of dexamethasone for 2-4 days, followed by administration of 12 mg/day of dexamethasone for 2-4 days, followed by administration of 8 mg/day of dexamethasone for 2-4 days, followed by administration of 6 mg/day of dexamethasone for 2-4 days, followed by administration of 4 mg/day of dexamethasone for 2-4 days, followed by administration of 2 mg/day of dexamethasone for 2-4 days. In this way, a starting baseline dosage of dexamethasone, which may be any dosage of dexamethasone known in the art including but not limited to 16-40 mg/day, may be tapered to a lower dose or a zero dose over time. The route, frequency and amount of composition administered may be adjusted by one of skill in the art based on any known guidelines for achieving the desired result. In one embodiment of the claimed method, the compositions of the invention are administered in a treatment regimen such that the symptoms of cerebral edema subside.

It should be noted that the amount of corticorelin acetate that the patient is administered depends on the amount of steroid that the patient receives. Also, throughout the period of time that the patient is receiving corticorelin acetate, the amount of corticorelin acetate can be increased in order to increase its steroid-sparing effect, i.e. the dose of corticorelin acetate can increase over time as the dose of steroid that the patient receives decreases over time. In alternative embodiments, the amount of corticorelin acetate that the patient receives may stay constant as the dose of steroid that the patient receives decreases. Also, the amount of corticorelin acetate that a patient is administered can be adjusted based on the increase or decrease of steroid-associated side-effects.

In certain embodiments, the patient is receiving dexamethasone at a dosage of between 0.75 and 9 mg per day, prior to the patient's initial administration of corticorelin acetate. In one embodiment, the patient has been diagnosed with a recurrent or inoperable brain tumor and is receiving a 2 mg dose of dexamethasone two to three times per day. In another embodiment, the patient has been diagnosed with cerebral edema and is administered an initial dose of 10 mg dexamethasone intravenously, followed by 4 mg every six hours intramuscularly until the symptoms of cerebral edema subside.

In certain embodiments, the corticorelin acetate when administered in an amount to provide a steroid-sparing effect may eliminate the side-effects associated with the steroid that the patient experiences. In other embodiments, the methods described herein include methods for steroid-sparing treatment in a human in need thereof comprising administering to the human a combination of a sub-clinically effective amount of steroid and corticorelin acetate.

In certain methods of the invention, administration of corticorelin acetate to a patient eliminates the need for a patient to take additional medications, such as, for example, antibiotics or anti-ulcer agents. Accordingly, the methods of the invention encompass a method for reducing the frequency or dosage of antibiotics administered to a patient comprising administering to a patient that is receiving steroid treatment an amount of corticorelin acetate such that the frequency or dosage of steroid administered to the patient is reduced over time, and wherein the frequency or dosage of antibiotics administered to the patient is reduced over time. In one embodiment, the dosage of antibiotics is reduced to zero. Antibiotics of the invention are known in the art and include but are not limited to aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin. The methods of the invention also encompass a method for reducing the frequency or dosage of an anti-ulcer agent administered to a patient comprising administering to a patient that is receiving steroid treatment an amount of corticorelin acetate such that the frequency or dosage of steroid administered to the patient is reduced over time, and wherein the frequency or dosage of anti-ulcer agent administered to the patient is reduced over time. In one embodiment, the dosage of anti-ulcer agent is reduced to zero. Anti-ulcer agents are known in the art and include but are not limited to belladonna alkaloids, synthetic anticholinergic agents, lansoprazole, omeprazol, rabeprazole, famotidine, cimetidine, ranitidine hydrochloride, prostaglandins such as 17,20-dimethyl-6-oxoprostaglandin-E₁ methyl ester, 15-methyl-prostaglandin E₂, 16-methyl-16-hydroxy-15-dehydroxyprostaglandin E₁ methyl ester, 7-thiaprostaglandin E₁ methyl ester, and 17,20-dimethyl-7-thiaprostaglandin E₁ methyl ester.

Additionally, the present invention includes methods of providing replacement therapy for steroid therapy in a patient receiving such therapy comprising administration of a steroid-sparing amount of corticorelin acetate. In certain embodiments, corticorelin acetate can be administered to a patient as an alternative to a steroid. In certain embodiments, steroid replacement therapy comprising administration of corticorelin acetate can be administered to refractory patients. For example, the present invention includes administration of corticorelin acetate to patients refractory to steroid therapy. The methods of the invention also comprise administering dexamethasone to a patient that is concurrently receiving steroid and anti-diabetic medication. Anti-diabetic agents are well-known in the art.

### 5.3.4 Dose-Sparing of Additional Agents Administered for the Treatment of Inflammation or Cancer

Through the present invention is directed to methods of providing a steroid-sparing benefit to a human that is presently receiving a steroid comprising administering corticorelin acetate, the present invention also encompasses methods of dose-sparing of an anti-inflammatory or an anti-cancer agent in a human in need thereof comprising administering to the human an amount of corticorelin acetate, wherein the amount of the corticorelin acetate is sufficient to allow the effect of the anti-inflammatory or an anti-cancer agent to be attained in a lower amount than in the absence of the corticorelin acetate.

Anti-inflammatory agents include, but are not limited to, diuretics such as loop diuretics, osmotic diuretics proximal diuretics, distal convoluted tubule diuretics and cortical collecting tubule diuretics. For example, suitable diuretics include, but are not limited to, glucose, mannitol, bumetanide, ethacrynic acid, furosemide, torsemide, amiloride, spironolactone, triamterene, bendroflumethiazide, hydrochlorothiazide, acetazolamide, dorzolamide, phosphodiesterase, chlorthalidone, caffeine, metolazone and combinations thereof.

Anti-cancer agents include, but are not limited to, anti-neoplastic, anti-proliferative, anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins. Other anti-cancer agents include carboplatin, Temodar® (temozolomide), 6-Benzylguanine, lapatinib (GW572016), topotecan, bevacizumab, and irinotecan (Camptosar).

When corticorelin acetate is administered with anti-inflammatory or anti-cancer agents the corticorelin acetate and the anti-cancer or anti-inflammatory agent can be administered sequentially, cyclically or simultaneously. If administered sequentially, the order of administration is flexible.

### 5.4 Target Patient Populations

In accordance with the invention, corticorelin acetate can be administered to patients diagnosed with edema. In certain embodiments, the methods of the invention comprise administering corticorelin acetate to patients who are diagnosed with brain edema to reduce, stabilize, or slow the progression of the edema. Patients with brain edema include patients with intracellular or extracellular brain edema. Intracellular edema includes cytotoxic edema. Extracellular edema includes vasogenic edema, hydrocephalic edema, osmotic edema, statsis induced by tumor in venous drainage, hydrodynamic processes in which fluid originates from a tumor, and excretory-secretory mechanisms in meningiomas. Accordingly, in one embodiment, the methods of the invention encompass administering corticorelin acetate to a patient to reduce the risk of developing edema.

In other embodiments patients administered corticorelin acetate are patients that are at risk of developing edema due to the fact that the homeostatic balance of interstitial fluid is disrupted. Methods of monitoring the homeostatic balance of interstitial fluid are known in the art and include clinical and neurological testing methods discussed in Section 5.3.3, infra.

Corticorelin acetate can be administered to patients who have been diagnosed with brain edema or who are at risk of developing brain edema as the result of trauma, injury, toxins or disease to the brain such as traumatic head injuries, craniotomies, blood clots, brain cysts, inflammation or infections. Patients diagnosed with brain edema or who are at risk of developing brain edema due to mechanical, circulatory, osmotic, or metabolic disruptions can also be administered corticorelin acetate.

The invention also encompasses administering corticorelin acetate to patients who have brain tumors or patients suffering from recurrent brain tumors who are also diagnosed with or at risk of developing brain edema. Such brain tumors can be primary or metastatic. The present invention also includes administration of corticorelin acetate to patients anticipating or preparing to have surgery, currently undergoing surgery or recovery from surgery associated with a brain tumor. In certain embodiments patients anticipating or preparing for resection i.e. surgical removal of a brain tumor, currently undergoing resection or recovering from resection can be administered corticorelin acetate.

Additionally, patients anticipating or planning to receive chemotherapy, currently receiving chemotherapy or patients recovering from chemotherapy may be at risk for developing or may be diagnosed with edema. Accordingly in one embodiment, the methods of the invention encompass administering corticorelin acetate to a patient planning to receive chemotherapy, currently receiving chemotherapy or patients recovering from chemotherapy. Moreover, patients suffering from edema associated with side-effects of surgery or chemotherapy can be administered corticorelin acetate.

Additionally, patients anticipating or planning to receive radiotherapy, currently receiving radiotherapy or patients recovering from radiotherapy may be at risk for developing or may be diagnosed with edema. Accordingly in one embodiment, the methods of the invention encompass administering corticorelin acetate to a patient planning to receive radiotherapy, currently receiving radiotherapy or patients recovering from radiotherapy. Moreover, patients suffering from edema associated with side-effects of surgery or radiotherapy can be administered corticorelin acetate. For example, patients diagnosed with brain edema with radiation necrosis can be administered corticorelin acetate.

Also in accordance with the invention, corticorelin acetate can be administered to patient who is also receiving a steroid or who is undergoing steroid therapy. In some embodiments of the invention, the patient has undergone steroid treatment for a prolonged period of time. Accordingly, methods of the invention comprise administering corticorelin acetate to a patient that has been receiving steroid therapy for over 1 month, over 6 months, over 1 year, or over 2 years. In other embodiments, the patient is receiving high doses of steroid treatment. In one embodiment the patient is receiving 20-24 mg/d of steroid. In certain embodiments, corticorelin acetate is administered to patients suffering from neurological or clinical decline as a result of the steroid treatment. Neurological or clinical decline can be measured by any method known in the art including, but not limited to, clinical and neurological tests. Clinical tests that may be used with the methods of the invention include medical imaging, such as CT scans, MRI and measurement of serum cortisol levels. Neurological tests include NCI neurological exam, Karnofsky Performance Status (KPS) and mini-mental status examination (MMSE) scores.

Additionally, the methods and compositions of the invention are useful in the treatment of patients who have unsuccessfully attempted to stop steroid treatment or patients who have unsuccessfully attempted to reduce the dosage or frequency of administration of the steroid.

Corticorelin acetate can also be administered to patients experiencing unwanted side-effects associated with the steroid treatment. Side-effects associated with steroid therapy are discussed in Section 5.3.1, infra.

In another aspect of the present invention, the methods and compositions of the invention are useful in the treatment of a patient adverse or refractory to steroid therapy. In certain embodiments patients who are diagnosed with brain edema and who are refractory to steroids are administered corticorelin acetate. In certain embodiments, patients refractory to steroid therapy are patients who have been administered a steroid for the treatment of a disease, such as edema, and wherein the disease being treated i) showed no response to the steroid therapy for a period of time, or ii) did exhibit a response but at some point during the steroid therapy ceased to exhibit a response, or iii) the disease proceeded to advance. Methods to determine whether a patient is refractory to steroid treatment are known in the art. For example, assessment of whether a patient is refractory to steroid therapy can be determined by clinical or neurological tests. Clinical tests including medical imaging such as CT scans and MRI; measurement of serum cortisol levels. Neurological tests include NCI neurological exam, Karnofsky Performance Status (KPS) and mini-mental status examination (MMSE) scores. In certain embodiments patients diagnosed with brain edema and who are 55 years or older and refractory to steroids can be administered corticorelin acetate.

### 5.6 Dosing Regimens

In any of the above described methods, corticorelin acetate can be administered once a day or multiple times a day. For example, the dosages of corticorelin acetate can be administered every hour, every two hours, every three hours, every four hours, every six hours, every eight hours or every 12 hours. Alternatively, corticorelin acetate can be administered once every two, three, four, five or six days. In certain embodiments corticorelin acetate can be administered once a week, once every two, three or four weeks or once a month.

Additionally, as shown in the examples below, corticorelin acetate has been shown to be well tolerated when administered over long periods of time. Therefore, a patient who is administered corticorelin acetate can be placed on a dosing regimen wherein the patient receives corticorelin over an extended period of time. In certain embodiments, the patient can receive administrations of corticorelin acetate over a period of 1 week, 2 weeks, 3 weeks, 4 weeks or more. In still other embodiments a patient can receive corticorelin acetate over a period of 1 month, 2 months, 3 months, 4 months, 5 months, 6 months or more. In some instances the patient can receive corticorelin acetate over a period of 1 year or longer. Ideally, a patient is administered corticorelin acetate until the edema no longer exists.

In any of the methods described above, the total daily dose of corticorelin acetate can range from 1 μg to 10 mg. In certain embodiments the total daily dose of corticorelin acetate can be 0.1 mg to 5 mg, or 0.3 mg to 2 mg. For example, the total daily dose of corticorelin acetate can be about 0.3 mg, about 0.5 mg, about 1 mg, about 2 mg, about 4 mg or about 5 mg. Corticorelin acetate can be administered once a day or multiple times a day until the desired daily dose of corticorelin acetate is reached. For example, 0.5 mg or 1.0 mg of corticorelin acetate can be administered 2 times a day to achieve a total daily dose of 1 mg or 2 mg of corticorelin acetate. Alternatively, 0.5 mg or 1.0 mg of corticorelin acetate can be administered 4 times a day to achieve a total daily dose of 2 mg or 4 mg of corticorelin acetate.

The invention further provides methods for improving the therapeutic outcome of a steroid comprising administering corticorelin acetate, in conjunction with the administration of the steroid. In one embodiment, the invention encompasses methods wherein the administration of a steroid with corticorelin acetate has additive potency or additive therapeutic effect. The invention also encompasses synergistic outcomes where the therapeutic efficacy is greater than additive. Preferably, such administration of a steroid with corticorelin acetate also reduces or avoids unwanted or adverse effects. Given the invention, in certain embodiments, doses of steroid can be reduced or administered less frequently, preferably increasing patient compliance, improving therapy and/or reducing unwanted or adverse effects. In a specific embodiment, lower or less frequent doses of steroid are administered to reduce or avoid unwanted effects if the steroid is administered with corticorelin acetate.

The corticorelin acetate can be administered over a period of time which may precede, overlap, and/or follow a treatment regimen with a steroid. Corticorelin acetate can be administered concurrently, before, or after the administration of the steroid. In one embodiment, the present invention provides a method comprising administering corticorelin acetate to the patient before, concurrently with, or after the administration of the steroid. In a specific embodiment, corticorelin acetate can augment the therapeutic benefit of a steroid and improve the outcome of the treatment. In a specific embodiment, corticorelin acetate is administered before the administration of the steroid. In another specific embodiment, the steroid is administered before the administration of corticorelin acetate. In other embodiments, corticorelin acetate is initially administered to a patient concurrently with the initial administration of a steroid. In a preferred embodiment, the steroid is dexamethasone.

In certain embodiments, the administration of corticorelin acetate in the absence of administration of the steroid or the administration of the steroid in the absence of administration of corticorelin acetate is not therapeutically effective. In a specific embodiment, the amount of corticorelin acetate or steroid is administered in an amount insufficient to be therapeutically effective alone. In alternate embodiments, both or at least one of the corticorelin acetate or steroid is therapeutically effective when administered alone.

In various specific embodiments, the above methods comprise the administration of corticorelin acetate to a patient treated with a steroid wherein the steroid administered alone is not clinically adequate to treat the patient such that the patient needs additional effective therapy, e.g., a patient is unresponsive to a steroid without administering corticorelin acetate. Included in such embodiments are methods comprising administering corticorelin acetate to a patient receiving a steroid wherein said patient has responded to therapy yet suffers from side effects, relapse, develops resistance, etc. Such a patient might be non-responsive or refractory to treatment with the steroid alone. The embodiments provide that the methods of the invention comprising administration of corticorelin acetate to a patient refractory to steroid alone can improve the therapeutic effectiveness of the steroid when administered as contemplated by the methods of the invention.

In a specific embodiment, corticorelin acetate is administered to a patient receiving a steroid for the treatment of edema wherein the patient may be non-responsive or refractory to treatment with the steroid alone, e.g., the edema has progressed. The determination of the effectiveness of a steroid can be assayed in vivo or in vitro using methods known in the art and using methods discussed in Section 5.3.3, infra. In one embodiment, the edema is refractory or non-responsive where the volume of interstitial fluid has not significantly reduced, or has increased over a period of time. Various method of identifying a patient that is refractory or non-responsive are well known in the art, including methods of monitoring in Section 5.3.3.

In certain specific embodiments, corticorelin acetate is administered to a patient already receiving steroid therapy (e.g., dexamethasone administered 1-24 mg daily). In such embodiments, corticorelin acetate is initially administered to a patient who has already been receiving dexamethasone in the absence of corticorelin acetate 2 days, 2 days to 1 week, 1 week to 1 month, 1 month to 6 months, 6 months to 1 year prior to administration of corticorelin acetate in addition to dexamethasone. In a specific embodiment, corticorelin acetate is administered to a patient wherein the patient showed resistance to treatment with dexamethasone alone.

Corticorelin acetate can also be administered as a steroid-sparing agent to facilitate steroid taper. For instance, corticorelin acetate can be administered to a patient, preferably a human that is already receiving steroid therapy wherein upon receiving the corticorelin acetate the steroid dose that the patient is receiving can be reduced. In certain embodiments, a patient who is currently receiving dexamethasone can be administered corticorelin acetate wherein corticorelin acetate is administered to a patient to facilitate steroid taper and the amount of dexamethasone that the patient is receiving can be reduced. For example, a patient who is receiving 1 mg/d, 2 mg/d, 3 mg/d, 4 mg/d, 5 mg/d, 6 mg/d, 7 mg/d, 8 mg/d, 9 mg/d, 10 mg/d, 11 mg/d, 12 mg/d, 13 mg/d, 14 mg/d, 15 mg/d, 16 mg/d, 17 mg/d, 18 mg/d, 19 mg/d, 20 mg/d, 21 mg/d, 22 mg/d, 23 mg/d or 24 mg/d of dexamethasone can be administered an amount of corticorelin acetate to facilitate steroid taper such that the amount of dexamethasone that the patient receives can be reduced to less than 1 mg/d, 2 mg/d, 3 mg/d, 4 mg/d, 5 mg/d, 6 mg/d, 7 mg/d, 8 mg/d, 9 mg/d, 10 mg/d, 11 mg/d, 12 mg/d, 13 mg/d, 14 mg/d, 15 mg/d, 16 mg/d, 17 mg/d, 18 mg/d, 19 mg/d, 20 mg/d, 21 mg/d, 22 mg/d, 23 mg/d or 24 mg/d and in some embodiments to 0 mg/day of dexamethasone.

In certain embodiments, as the amount of steroid administered to a patient decreases, the amount of corticorelin acetate increases. For example, a patient receiving 6 mg/d of dexamethasone can be administered 1 mg/d of corticorelin acetate for three days. At the end of three days the patient can receive 4 mg/day of dexamethasone and 2 mg/day of corticorelin acetate for three additional days. At the end of day six the patient can be administered 4 mg/day of dexamethasone and 3 mg/day of corticorelin acetate for an additional three days.

In other embodiments, as the amount of steroid the patient received decreases, the amount of corticorelin administered remains constant. For example, a patient receiving 6 mg/d of dexamethasone can be administered 1 mg/d of corticorelin acetate for three days. At the end of three days the patient can receive 2 mg/day of dexamethasone and 1 mg/day of corticorelin acetate for three additional days. At the end of day six the patient can be administered 4 mg/day of dexamethasone and 1 mg/day of corticorelin acetate for an additional three days.

In still yet other embodiments, as the amount of steroid the patient receives decreases, the amount of corticorelin acetate administered can also decrease. In any of the above embodiments, once the amount of the steroid the patient is administered reaches 0 mg/d, the amount of corticorelin acetate that patient receives can also be decreased.

In a specific embodiment, corticorelin acetate is administered to a patient receiving a steroid for the treatment of edema wherein the patient may experience unwanted or adverse effects to treatment with the steroid alone, e.g., the steroid may be toxic or harmful at its effective dose, administered alone. Given the invention, corticorelin acetate can improve the therapeutic benefit of the steroid such that the dosage or frequency of administration of the steroid can be lowered when administered in conjunction with corticorelin acetate. In a preferred embodiment, corticorelin acetate is administered to a patient to reduce or avoid the unwanted or adverse effects of the steroid alone, wherein the administration of corticorelin acetate allows lower and/or less frequent doses of the steroid.

In various embodiments, such as those described above, the corticorelin acetate and steroid are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart, or no more than 1 week or 2 weeks or 1 month or 3 months apart. In other embodiments, corticorelin acetate and steroid are administered 2 to 4 days apart, 4 to 6 days apart, 1 week apart, 1 to 2 weeks apart, 2 to 4 weeks apart, one month apart, 1 to 2 months apart, or 2 or more months apart. In preferred embodiments, corticorelin acetate and steroid are administered in a time frame where both are still active. One skilled in the art would be able to determine such a time frame by determining the half life of each administered component. In the foregoing embodiments, the corticorelin acetate and steroid are administered less than 2 weeks, one month, six months, 1 year or 5 years apart. Preferably, the steroid is administered prior to corticorelin acetate.

In one embodiment, corticorelin acetate and the steroid are administered within the same patient visit. In one embodiment, the steroid is administered prior to the administration of corticorelin acetate. In an alternate embodiment, the steroid is administered subsequent to the administration of the steroid.

In certain embodiments, the corticorelin acetate and steroid are cyclically administered to a patient. Cycling therapy involves the administration of the steroid for a period of time, followed by the administration of corticorelin acetate for a period of time and repeating this sequential administration. Cycling therapy can reduce the development of resistance to the steroid, avoid or reduce the side effects of steroid therapy, and/or improve the efficacy of the treatment. In such embodiments, the invention contemplates the alternating administration of a steroid followed by the administration of corticorelin acetate. In certain embodiments, the corticorelin acetate and steroid are alternately administered in a cycle of less than 3 weeks, once every two weeks, once every 10 days or once every week.

In other embodiments, the corticorelin acetate and steroid are alternately administered in cycles of less than 3 weeks, once every two weeks, once every 10 days or once every week.

Additionally, corticorelin acetate can be administered to a patient that may have had surgery, or recovering from surgery, or currently undergoing surgery, or anticipates undergoing surgery. Such surgery can be associated with treatments for cancer; brain tumors such as resection; or edema such as brain edema.

Corticorelin acetate can also be administered in conjunction with an additional anti-cancer or anti-inflammatory treatment to reduce the side-effects of such therapy. Suitable anti-inflammatory treatments include, but are not limited to, administration of an anti-inflammatory agent. Anti-inflammatory agents include, but are not limited to, diuretics such as those discussed in Section 5.3.4.

Anti-cancer treatments include radiotherapy, chemotherapy, photodynamic therapy, surgery or other immunotherapy. Chemotherapy can include the administration of anti-neoplastic, anti-proliferative, anti-miotic agents such as those discussed in Section 5.3.4.

In another preferred embodiment, corticorelin acetate is administered to a patient receiving radiation therapy for treatment of cancer. For radiation treatment, the radiation can be gamma rays or X-rays. The methods encompass administering corticorelin acetate to a patient receiving radiation therapy, such as external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management Radiation Therapy, 6th edition, 2001, DeVita et al., eds., J.B. Lippencott Company, Philadelphia.

In certain embodiments, individuals receiving radiotherapy, chemotherapy, photodynamic therapy, surgery or other immunotherapy or chemotherapy treatments for cancer and who have edema or at risk of developing edema can be administered an effective amount of corticorelin acetate for the treatment of the edema in conjunction with the radiotherapy, chemotherapy, photodynamic therapy or surgery.

When corticorelin acetate is administered with anti-inflammatory or anti-cancer agents the corticorelin acetate and the anti-cancer or anti-inflammatory agent can be administered sequentially or simultaneously. If administered sequentially, the order of administration is flexible.

### 5.7 Administration

The above methods include dosing regimens comprising corticorelin acetate. Preferably in any of the methods discussed above, the corticorelin acetate is administered by subcutaneous injection, such as subcutaneous bolus injection. The inventors have achieved unexpected results that include therapeutically effective treatment regimens for managing edema as well as steroid-sparing effects of corticorelin acetate when corticorelin acetate is administered as a subcutaneous bolus injection. Moreover, in one embodiment subcutaneous injection is preferred over additional routes of administration such as intravenous routes of administration due to the fact that administration of subcutaneous corticorelin acetate would require less frequent administration of corticorelin acetate, making administration less of a burden on the patient, and thereby increasing patient compliance, improving therapy. In other embodiments, subcutaneous administration may result in fewer side-effects that are associated with intravenous infusion such as, but not limited to, headache, hypotension, facial flushing, diarrhea, shortness of breath, nausea and lethargy.

In certain embodiments, corticorelin acetate described herein is administered by subcutaneous injection in an amount of 0.1 μg/kg to 1000 μg/kg. Corticorelin acetate can be administered subcutaneously in an amount of 1 μg/kg to 500 μg/kg, 2 μg/kg to 100 μg/kg, 2 μg/kg to 80 μg/kg, 4 μg/kg to 40 μg/kg, or 5 μg/kg to 20 μg/kg. For example, corticorelin acetate can be administered in 10 μg/kg, 30 μg/kg, 60 μg/kg, 100 μg/kg and 300 μg/kg doses.

In other embodiments, corticorelin acetate described herein can be administered by subcutaneous injection in an amount of 1 μg to 100 mg. Corticorelin acetate can be administered subcutaneously in an amount of 1 μg to 80 mg, 10 μg to 50 mg, 100 μg to 40 mg, 300 μg to 10 mg, 600 μg to 1 mg, and 800 μg to 1 mg. For example, corticorelin acetate can be administered subcutaneously in 100 μg, 300 μg, 600 μg, 1 mg, 2 mg, 4 mg and 5 mg doses.

Corticorelin acetate administered subcutaneously can be administered once a day or multiple times a day. For example, the dosages of corticorelin acetate administered subcutaneously can be administered every hour, every two hours, every three hours, every four hours, every six hours, every eight hours or every 12 hours. Alternatively, corticorelin acetate can be administered once every two, three, four, five or six days. In certain embodiments corticorelin acetate can be administered once a week, once every two, three or four weeks or once a month. Dosages of corticorelin acetate that are administered once a week or longer can be administered in the form of a depot. For example, the present invention includes methods of managing or treating brain edema comprising administering to a patient, preferably a human, in need thereof a therapeutically effective amount of corticorelin by subcutaneous injection. In certain embodiments, a patient diagnosed with brain edema is administered a subcutaneous injection of 1 mg of corticorelin acetate twice daily.

Though subcutaneous administration of corticorelin acetate is preferred, corticorelin can also be administered by other parenteral routes of administration such as, but not limited to, intradermal and intramuscular injections, and intravenous or intraosseous infusions. For example, corticorelin acetate can be administered by intravenous infusion in an amount of 0.1 μg/kg/h to 100 μg/kg/h. For example, corticorelin acetate can be administered intravenously in an amount of 1 μg/kg/h to 100 μg/kg/h, or 2 μg/kg/h to 80 μg/kg/h, or 2 μg/kg/h to 50 μg/kg/h, or 4 μg/kg/h to 40 μg/kg/h, or 5 μg/kg/h to 20 μg/kg/h.

In other embodiments corticorelin acetate can be administered intravenously in an amount of 1 μg/kg to 1000 μg/kg. For example corticorelin acetate can be administered intravenously in an amount of 1 μg/kg to 100 μg/kg, or 2 μg/kg to 80 μg/kg, or 2 μg/kg to 50 μg/kg, or 4 μg/kg to 40 μg/kg, or 5 μg/kg to 20 μg/kg. For example, corticorelin acetate can be administered in 0.5 μg/kg to 1 μg/kg, or 2 μg/kg to 8 μg/kg, or 4 μg/kg to 8 μg/kg, or 5 μg/kg doses.

Corticorelin acetate can be administered intravenously over a period of an hour or less than an hour. In certain embodiments corticorelin acetate can be administered intravenously over a period of one hour or more. For example, the dosages of corticorelin acetate administered intravenously, discussed above can be administered over a period of 10 min., 30 min., 45 min., one hour, two hours, four hours, eight hours, 12 hours, 24 hours, 48 hours or 72 hours.

In certain embodiment the dosing regimens comprises administering corticorelin acetate with a steroid, such as the steroids discussed in Section 5.3.1. The corticorelin acetate and the steroid can be administered sequentially or simultaneously. If administered sequentially, the order of administration is flexible. In a specific embodiment, corticorelin acetate is administered subcutaneously. In another specific embodiment, the steroid, such as dexamethasone is administered orally.

### 5.8 Pharmaceutical Compositions

The present invention relates to pharmaceutical compositions containing corticorelin acetate as the active ingredient to be administered in accordance with the methods described herein. The corticorelin acetate may be formulated with a pharmaceutically acceptable carrier. The pharmaceutical formulations of the present invention can take the form of solutions, suspensions, emulsions that include corticorelin acetate, and a pharmaceutically acceptable diluent, adjuvant or carrier. In certain embodiments, the pharmaceutical formulations of the present invention are formulated for subcutaneous bolus injection.

Pharmaceutical formulations comprising corticorelin acetate formulated for subcutaneous injection provided for treatment of edema and compared to corticorelin acetate formulations formulated for other types of parenteral administration can offer certain advantages. In certain embodiments, administration of subcutaneous formulations of corticorelin acetate can result in less frequent administration of corticorelin acetate than administration of other non-subcutaneous formulations of corticorelin acetate. Less frequent administration of corticorelin acetate can result in greater patient compliance. Additionally, in other embodiments, administration of subcutaneous formulations of corticorelin acetate can result in fewer side-effects associated with administration of non-subcutaneous formulations of corticorelin acetate.

In addition to subcutaneous formulations of corticorelin acetate, the pharmaceutical formulations of the invention may be formulated for other types of parenteral administration, including, but not limited to, intradermal and intramuscular injections, and intravenous or intraosseous infusions. The pharmaceutical formulations of the present invention can take the form of solutions, suspensions, emulsions that include corticorelin acetate, and a pharmaceutically acceptable diluent, adjuvant or carrier, depending on the route of administration.

The pharmaceutical compositions of the invention are formulated to deliver a therapeutic dose of corticorelin acetate of the invention. The dose of corticorelin acetate contained in a pharmaceutical formulation can range from 1 μg to 10 mg. In certain embodiments the dose of corticorelin acetate can range from 0.1 mg to 5 mg, or 0.3 mg to 2 mg. In certain embodiments, the dose of corticorelin acetate can be about 0.3 mg, about 0.5 mg, about 1 mg, about 2 mg, about 4 mg or about 5 mg. The doses can be determined by methods known in the art and the pharmaceutical formulations of the present invention can be administered alone or in combination to achieve i) a reduction in or stabilization of clinical or neurological side effects that may accompany steroid treatment for edema; or ii) a reduced exposure to steroids as compared to the exposure to steroid that would have been required to achieve the same or better therapeutic results in the absence of corticorelin acetate or in the absence of corticorelin acetate administered subcutaneously, as determined by methods known in the art.

The present invention is also directed to methods of treating edema by administering to a patient in need thereof corticorelin acetate and an additional therapeutic agent. The additional therapeutic agent can be any agent that can alleviate edema or when in combination with the corticorelin acetate can improve the effect of the additional therapeutic agent on the edema.

Suitable additional therapeutic agents include anti-inflammatory agents such as, but not limited to, corticosteroids, diuretics; anti-neoplastic; anti-proliferative; and anti-miotic agents. Examples of corticosteroids, diuretics; anti-neoplastic; anti-proliferative; and anti-miotic agents are discussed in Sections 5.3.1 and 5.3.4. Additionally, corticorelin acetate described herein can be co-administered with other anti-cancer treatments such as, radiotherapy, chemotherapy, photodynamic therapy, surgery or other immunotherapy.

The corticorelin acetate and the additional therapeutic agent can be administered sequentially, cyclically or simultaneously. If administered sequentially, the order of administration is flexible. For instance, the corticorelin acetate can be administered prior to administration of the additional therapeutic agent. Alternatively, administration of the additional therapeutic agent can precede administration of corticorelin acetate.

Whether they are administered as separate compositions or in one composition, each composition is preferably pharmaceutically suitable for administration. Moreover, corticorelin acetate and the additional therapeutic agent, if administered separately, can be administered by the same or different modes of administration.

### 6.0 EXAMPLES

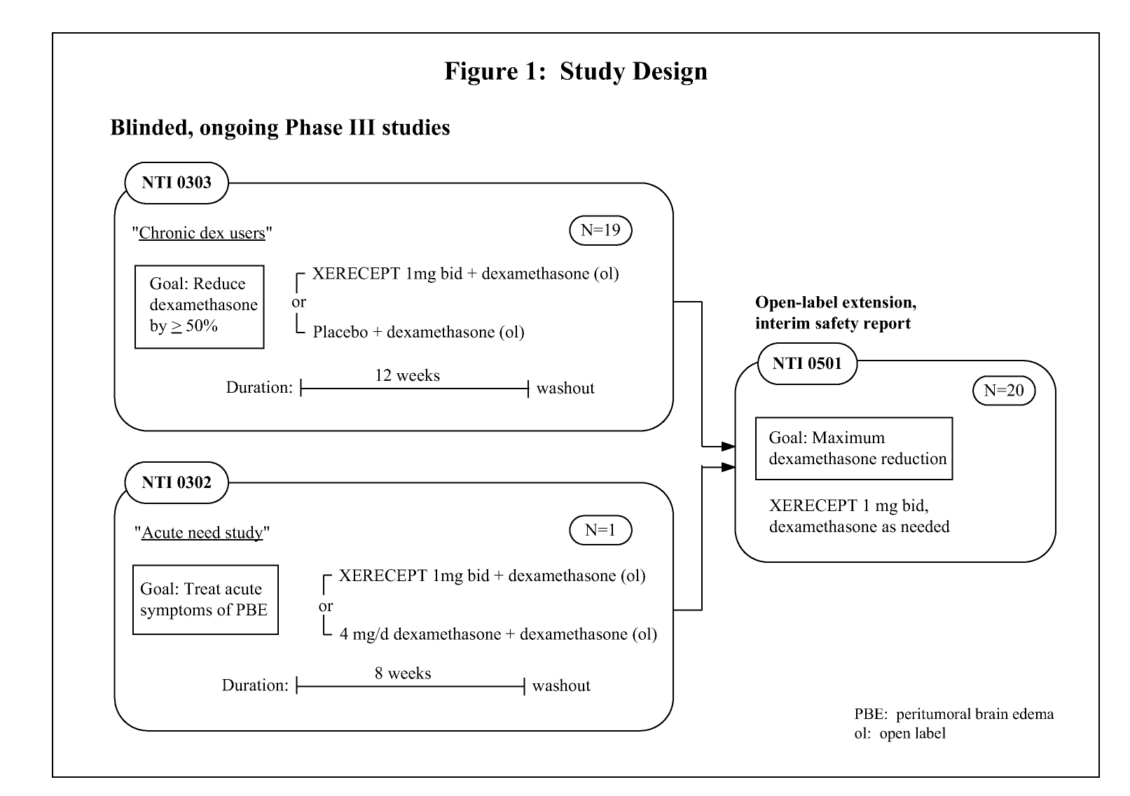

Eligible patients with primary or secondary brain tumors, who had previously participated in one of two randomized, double-blind Phase III trials, were then enrolled in an open-label extension study. The design of the extension study is shown in FIG. 1. As shown in FIG. 1 patients that participated in the Phase III studies that received XERECEPT (corticorelin acetate) 1 mg twice daily and dexamethasone or placebo and dexamethasone or dexamethasone alone were enrolled in the open label extension study. One of the goals of the open label study was to study the maximum dexamethasone reduction in each patient.

In the open-labeled extension study, patients returned at 4-week intervals and completed assessments that included adverse events, dexamethasone dose, and steroid side-effects. During the study, dexamethasone was maximally reduced as tolerated but corticorelin acetate dose was not increased. Net cumulative change in dexamethasone dose (NCD) for each patient was determined by the change in dexamethasone dose from baseline for each visit. The net sum of these changes accounted for duration, direction, and amount of dexamethasone dose change.

The demographic and baseline characteristics of the first twenty patients to take corticorelin acetate for more than 4 weeks in the extension study are shown in Table 1. Of the twenty patients reviewed 14 were men and 6 were women. The patients ranged in ages of 33 to 67 with a median age of 53. Prior to the randomized study, reduction of dexamethasone treatments were attempted in 19 of the 20 patients of the 19 patients, attempt to reduce the dexamethasone dose were unsuccessful in 16 (84%) patients.

### Table 1

TABLE 1
Demographics and Baseline Characteristics

| | | | | | Prior attempt | | |
|---|---|---|---|---|---|---|---|
| | | | | | to reduce dex | | |
| | | | | | Yes/No | | |
| | | | | | Successful (S) | Recurrent | Baseline |
| | | | Tumor | Tumor Dx | Unseccessful | Disease | Dexamethasone |
| ID # | Age | Gender | Diagnosis | Date | (U) | Yes/No | mg/d |
| 1 | 42 | M | Malignant | 09/1999 | YES - U | YES | 6.0 |
| | | | Astrocytoma | | | | |
| 2 | 53 | M | Anaplastic | 00/1994 | YES - U | YES | 6.0 |
| | | | Ganglioneuro- | | | | |
| | | | cytoma | | | | |
| 3 | 57 | M | GBM | 02/2005 | YES - U | NO | 8.0 |
| 4 | 46 | F | GBM | 10/2003 | YES - U | YES | 2.5 |
| 5 | 67 | M | Meningioma | 00/1969 | YES - U | NO | 4.0 |
| 6 | 58 | F | Occipital | 00/1969 | YES - U | NO | 6.0 |
| | | | Meningioma | | | | |
| 7 | 47 | F | Metastatic | 08/2003 | YES - U | YES | 8.0 |
| | | | Breast Cancer | | | | |
| 8 | 50 | M | GBM | 12/2003 | YES - U | YES | 12 |
| 9 | 33 | M | GBM | 10/2003 | YES - U | YES | 2.5 |
| 10 | 63 | M | GBM | 10/2005 | YES - S | NO | 4.0 |
| 11 | 65 | F | GBM | 05/2005 | YES - U | NO | 4.5 |
| 12 | 53 | F | Metastatic Non- | 08/2004 | YES - S | NO | 0 |
| | | | small | | | | |
| | | | Cell Lung | | | | |
| | | | Cancer | | | | |
| 13 | 39 | M | GBM | 08/2003 | YES - U | YES | 0 |
| 14 | 58 | M | GBM | 05/2004 | YES - U | NO | 8.0 |
| 15 | 47 | M | GBM | 09/2004 | YES - S | YES | 2.0 |
| 16 | 58 | M | Anaplastic | 03/1995 | YES - U | YES | 3.0 |
| | | | Oligodendroglioma | | | | |
| 17 | 53 | F | Metastatic Lung | 12/2004 | NO | NO | 3.0 |
| | | | Cancer | | | | |
| 18 | 55 | M | GBM | 11/2004 | YES - U | NO | 24.0 |
| 19 | 57 | M | Metastatic | 02/2003 | YES - U | YES | 4.0 |
| | | | Adeno | | | | |
| | | | Carcinoma | | | | |
| | | | Lung | | | | |
| 20 | 51 | M | GBM | 01/2000 | YES - U | YES | 0 |

At data extraction, these 20 patients had completed a median of 18.5 weeks (4 to 48 weeks) on corticorelin acetate 1.0 mg administered subcutaneously twice a day, except for 2 patients whose dose was reduced to 1.0 mg once daily after 12 or 20 weeks, respectively, because of stable improvements on twice daily dosing after discontinuation of dexamethasone. The patient whose dose was reduced after 12 weeks was taken off corticorelin acetate 8 weeks later because of continued stable improvement off dexamethasone.

Table 2 summarizes treatment-emergent adverse events (AEs) of the twenty patients studied.

TABLE 2
Table 2: Treatment-Emergent Adverse Events
Frequency and Severity ot Possibly or Probably Related AEs (n = 20)

| | Mild | Moderate | Severe | |
|---|---|---|---|---|
| Preferred Term | n (%) | n (%) | n (%) | n (%) |
| All adverse events | 3 (15.0) | 3 (15.0) | 1 (5.0) | 7 (35.0) |
| Eyelid edema | 1 (5.0) | | | 1 (5.0) |
| Photophobia | | 1 (5.0) | | 1 (5.0) |
| Nausea | | 1 (5.0) | | 1 (5.0) |
| Injection site erythema | 4 (20.0) | 1 (5.0) | | 5 (25.0) |
| Injection site reaction | 2 (10.0) | 1 (5.0) | | 3 (15.0) |
| Headache | | | 1 (5.0) | 1 (5.0) |
| Flushing | 3 (15.0) | 2 (10.0) | | 5 (25.0) |

Four patients died between 1-40 days after the last dose of corticorelin acetate, all because of conditions not related to study drug: progression of primary lung cancer (2 patients); progressive GBM (1 patient); deep vein thrombosis (1 patient). No patient reduced or discontinued corticorelin acetate because of a related AE. Eight patients had 12 SAEs, none of which were attributed to corticorelin acetate.

Serial serum cortisol levels were obtained for 16 patients, and were normal or low in 15 (94%) of the 16 patients. In one patient, AM serum cortisol (reference range 5-25 μg/dL) was low on a dexamethasone dose of 2.5 mg/d, moderately elevated (39 μg/dL) after discontinuation of dexamethasone for 4 weeks, then normalized (25 μg/dL) after 16 weeks off dexamethasone, and again low (0.4 μg/dL) after 8 weeks on dexamethasone at 7.0-8.0 mg/d.

Reduced NCD was observed in 11 (55%) of the 20 patients, while increased NCD was found in 7 (35%) of the 20 patients. Two (10%) of 20 patients took 0.0 mg/d dexamethasone from baseline to ≧24 weeks. In the 11 patients with reduced NCD, the median maximum percent change from baseline was −87% (−11 to −100%) with 5 patients discontinuing dexamethasone altogether. In the 7 patients with increased NCD, the median maximum percent change from baseline was +100% (+50 to +200%).

In the 11 patients whose NCD was reduced, the mean dexamethasone dose at enrollment was 6.2 mg/d; median 6.0 mg/d (2.5 to 12.0 mg/d). The mean NCD was −10.7 mg/d; the median NCD was −3.0 mg/d (−0.5 to −36 mg/d).

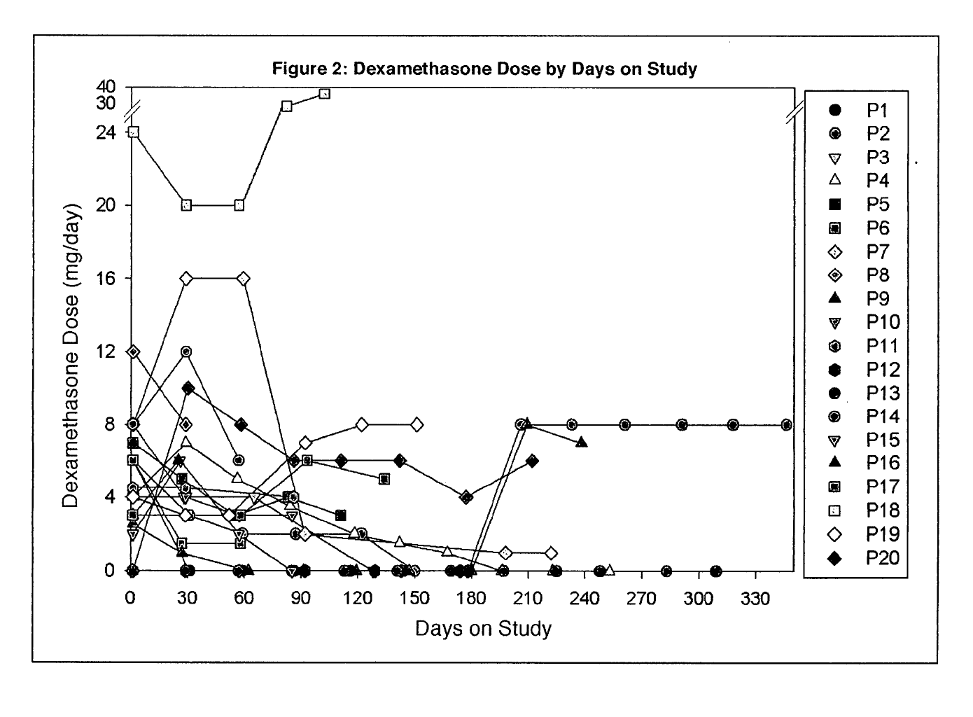

In the 7 patients whose NCD was increased, the mean dexamethasone dose at enrollment was 6.3 mg/d; median 3.0 mg/d (2.0 to 24.0 mg/d). The mean NCD was +10.7 mg/d; the median NCD was +5.0 mg/d (+2.0 mg/d to +46.0 mg/d). FIG. 2 presents dexamethasone dosing, over time for each of the 20 patients.

Improvement or resolution of 17 steroid-related conditions was observed in 8 (45%) of the 18 patients with such conditions at baseline. In these 8 patients, the mean NCD was −9.8 mg/d; the median NCD was −6.5 mg/d (0 to −36 mg/d).

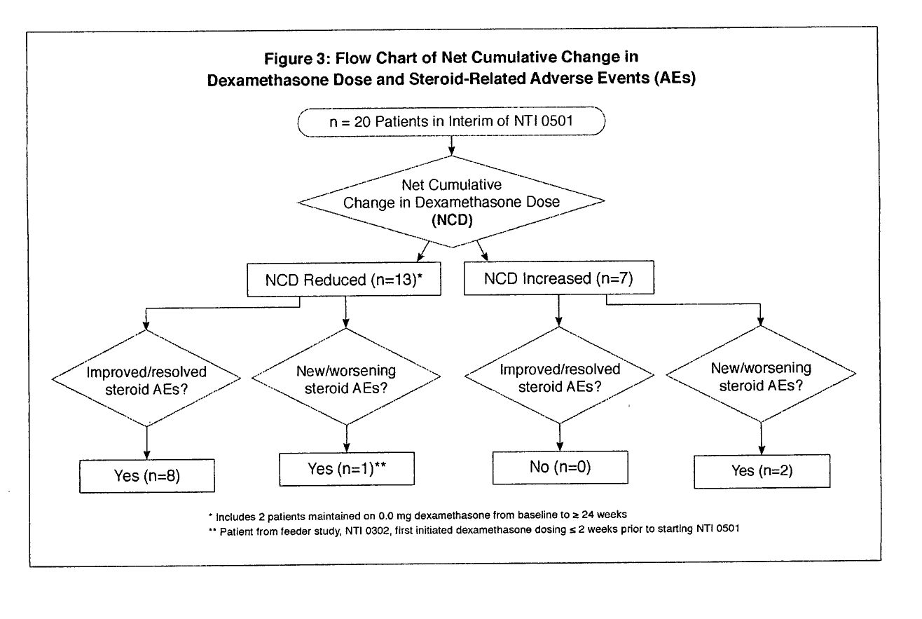

Mean baseline dexamethasone dose was similar in patients with reduced NCD (6.2 mg) and those with increased NCD (6.3 mg). Improvement or resolution of baseline steroid-related conditions correlated with NCD. Such improvement was observed only in patients whose NCD was reduced or those who took no dexamethasone from baseline to date extraction, as shown in FIG. 3. FIG. 3 is flow chart depicting the net cumulative change in dexamethasone dose and steroid-related adverse events. In contrast, worsening or new onset of steroid-related AEs was confined to patients whose NCD was increased, with one exception—a patient who initiated dexamethasone ≦2 weeks prior to baseline. Improvement was most frequently observed in Cushingoid appearance (5 AEs) myopathy (4 AEs), and insomnia (2 AEs) followed by weight gain, bloating, pedal edema, polyuria, skin striae, and Herpes zoster rash (1 AE each). Median time to initial improvement of steroid-related conditions was 12 weeks (1 to 36 weeks).

Typically refractory patients who successfully reduced dexamethasone included 5 (62.5%) of the 8 patients 55 years of age, 7 (70%) of the 10 patients with recurrent brain tumor, and 11 (69%) of the 16 patients in whom prior dexamethasone reductions had been unsuccessful.

Dexamethasone increases were prompted by neurologic worsening associated with a) progression of brain tumor (fatal in 2 patients); b) brain edema requiring surgical resection of tumor (dexamethasone was later decreased to 1.0 mg once daily because of neurologic improvement on corticorelin acetate); c) non-compliance with taking corticorelin acetate.

Long-term treatment with corticorelin acetate was safe and well-tolerated. Corticorelin acetate dosing up to 48 weeks did not induce hypercortisolism. Typically refractory patients whose dexamethasone dose was reduced included patients who were 55 years of age, had recurrent disease, or had failed previous attempts to dexamethasone reduction.

Improvement in steroid-related conditions was confined to patients whose NCD was reduced or those who took no dexamethasone during extended treatment with corticorelin acetate. New or worsening steroid-related AEs occurred in patients whose NCD was increased, and in one patient who initiated dexamethasone treatment within 2 weeks prior to baseline. Corticorelin acetate treatment was associated with reduced exposure to dexamethasone and improvement in steroid-related side-effects in patients with peritumoral brain edema.

The description and examples contained herein are for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

1. A method of treating brain edema in a patient in need thereof comprising administering a combination of corticorelin acetate and a steroid to a patient wherein the amount of the steroid in combination with corticorelin acetate is sufficient to reduce the edema.

2. The method of claim 1, wherein the steroid is a corticosteroid.

3. The method of claim 2, wherein the corticosteroid is dexamethasone.

4. The method of claim 1, wherein the corticorelin acetate and the dexamethasone are administered simultaneously.

5. The method of claim 1, wherein the corticorelin acetate and the dexamethasone are administered sequentially.

6. The method of claim 1, wherein the corticorelin acetate is administered subcutaneously.

7. The method of claim 1, wherein the corticorelin acetate is administered twice a day.

8. The method of claim 1, wherein the amount of corticorelin acetate administered is 1 mg.

9.-33. (canceled)

34. A method of treating a human with cancer comprising administering to a human an amount of corticorelin acetate, wherein the amount of the corticorelin acetate is sufficient to allow the effect of an anti-cancer agent to be attained in a lower amount than in the absence of the corticorelin acetate.

35. The method of claim 34, wherein the anti-cancer agent is bevacizumab.

36. A method of treating a human with a brain tumor comprising administering to a human corticorelin acetate in combination with an anti-cancer agent.

37. The method of claim 36, wherein the brain tumor is primary.

38. The method of claim 36, wherein the brain tumor is metastatic.

39. The method of claim 36, wherein the anti-cancer agent is bevacizumab.
